(19) United States
(12) Patent Application Publication
(10) Pub. No.: US 2010/0150870 A1
(43) Pub. Date: Jun. 17, 2010

(54) METHOD FOR IMPROVING THE SKIN AND COAT OF PETS

(75) Inventors: Linda A. Young, St. Joseph, MO (US); Terry Russell, Olean, MO (US); Jody Russell, Olean, MO (US)

Correspondence Address: K&L Gates LLP, P.O. Box 1135, CHICAGO, IL, 60690, US

(73) Assignee: NESTEC S.A., Vevey (CH)

(21) Appl. No.: 12/706,416

(22) Filed: Feb. 16, 2010

Related U.S. Application Data
(63) Continuation of application No. 10/070,799, filed on Sep. 16, 2002, now abandoned.

(51) Int. Cl. A61K 35/66; A23K 1/18

(52) U.S. Cl. 424/93.1; 426/2

(57) ABSTRACT

A method for improving or maintaining the skin and coat system of a pet includes administering to the pet a nutritional agent which promotes the growth of bifido- and lactic-bacteria in its gastro-intestinal tract. The nutritional agent may be a prebiotic or a probiotic micro-organism, or both. The nutritional agent may be administered together with a long chain fatty acid.

### CROSS REFERENCE TO RELATED APPLICATIONS

This application is a continuation of U.S. application Ser. No. 10/070,799, filed Sep. 16, 2002, which is a continuation of PCT/EP00/08747, filed Sep. 6, 2000, the entire contents of which are expressly incorporated herein by reference.

### FIELD OF THE INVENTION

This invention relates to a method of improving or maintaining the condition of the skin and coat system of a pet; especially dog and cat pets.

### BACKGROUND

Although the skin is the major external protective organ of a pet, a healthy coat is generally considered to be indicative of a healthy pet; and vice versa. For this reason, coat problems are one of the primary causes for pet owners to take their pets to veterinarians. These problems need not necessarily manifest themselves only in the coat, but may appear in the form of a dermatosis such as skin flaking, itchiness, skin lesions, and general dryness of the skin. These are often indicative of a malaise of the protective system provided by the skin and coat as an integrated functioning unit.

Much can be done to maintain or improve the pet's condition by feeding it a complete and balanced food. This provides the essential nutrients and minerals needed to maintain its skin and coat. However, in view of the many agents in the environment that may damage a pet's skin and coat system, feeding the pet a complete and balanced food may not always be sufficient. Therefore many supplements are commercially available for feeding to pets for improving their skin and coat. These supplements are often of questionable efficacy.

Zinc and linoleic acid are believed to improve the skin of pets. Therefore it has been suggested to include both zinc and linoleic acid in pet foods in excess of the pet's requirements. This approach is described in International Patent Application Number WO 98/56263. However, the levels of zinc required are in excess of those permitted in pet foods in certain markets. Also, high levels of linoleic acid are believed by some veterinarians to be pro-inflammatory.

Probiotic microorganisms are known from U.S. Pat. No. 57,560,088 to Matsuura et al to provide prophylaxis against dermatosis when administered in combination with a poly-unsaturated fatty acid and biotin and in the form of a prescription diet.

Despite these attempts, there nevertheless remains a need for a method of improving or maintaining the skin and coat system of pets in an integrated manner before the pet presents with disease symptoms. It is an object of this invention therefore to assist in alleviating this need.

### SUMMARY

This invention is based upon the finding that the condition of the skin and coat system of a pet may be improved, or at least maintained, by administering to the pet a nutritional agent which promotes the growth of bifido- and lactic-bacteria in the gastro-intestinal tract of the pet; and/or increases or improves the digestion of nutrients in the gastro-intestinal tract of the pet.

Accordingly, in one aspect, this invention provides a method of maintaining or enhancing the healthy functioning of the skin and coat system of a pet comprising the step of feeding the pet a food composition comprising a nutritional agent which promotes the growth of bifido- and lactic-bacteria in the gastro-intestinal tract of the pet. The healthy functioning may relate to the protective or appearance-enhancing role of the said system or to both.

The nutritional agent may be a prebiotic or a probiotic micro-organism, or may include both.

In a preferred embodiment, the nutritional agent is a prebiotic. A preferred example is inulin. The prebiotic is preferably derived from a plant source. The plant source may be included in the food composition. The composition may further include a probiotic micro-organism.

In a further aspect of the invention, a method for improving or maintaining the coat of a pet comprises administering to the pet a nutritional agent which promotes the growth of bifido- and lactic-bacteria in the gastro-intestinal tract of the pet. The nutritional agent preferably assists in improving the microflora balance on the skin of the pet.

In another aspect, this invention provides a method for improving or maintaining the skin and coat system of a pet, the method comprising administering to the pet a nutritional agent which increases the digestion of nutrients in the gastro-intestinal tract of the pet.

In a further aspect, this invention provides a method for improving or maintaining the skin and coat system of a pet, the method comprising administering to the pet a nutritional agent which improves the microflora balance on the skin of the pet.

The invention extends also to a method for improving or maintaining the shininess and softness of the coat of a pet, the method comprising administering to the pet a nutritional agent which promotes the growth of bifido- and lactic-bacteria in the gastro-intestinal tract of the pet.

Also provided, is a method of improving or maintaining shininess and softness of the coat of a pet, the method comprising administering to the pet a nutritional agent which increases the digestion of nutrients in the gastro-intestinal tract of the pet.

According to a further aspect, the invention provides a method of reducing or assisting in the prophylaxis of dandruff in the coat of a pet, the method comprising administering to the pet a nutritional agent which promotes the growth of bifido- and lactic-bacteria in the gastro-intestinal tract of the pet, or increases the digestion of nutrients in the gastro-intestinal tract of the pet.

Preferably the nutritional agent is administered to the pet in the form of a complete and nutritionally balanced pet food.

The nutritional agent may be a prebiotic or a probiotic micro-organism, or may include both in a complementary combination.

The pet food may further contain a source of long chain fatty acids, such as linoleic acid. In addition, it may contain a source of zinc.

In this specification:

“Prebiotic” means a substance or compound which is fermented by the intestinal flora of the pet and hence promotes the growth or development of bifido- and lactic-bacteria in the gastro-intestinal tract of the pet at the expense of pathogenic bacteria. The result of this fermentation is a release of fatty acids, in particular short-chain fatty acids in the colon. This has the effect of reducing the pH value in the colon.

“Probiotic micro-organism” means a micro-organism which beneficially affects a host by improving its intestinal microbial balance (Fuller, R; 1989; J. Applied Bacteriology, 66: 365-378). In general, probiotic micro-organisms produce organic acids such as lactic acid and acetic acid which inhibit the growth of pathogenic bacteria such as Clostridium perfringens and Helicobacter pylori.

Additional features and advantages are described herein, and will be apparent from the following Detailed Description and the figures.

### DETAILED DESCRIPTION

It has been surprisingly found that administering to a pet a nutritional agent which promotes the growth of bifido- and lactic-bacteria in the gastro-intestinal tract of the pet improves, or at least maintains, the condition of the skin and coat system of the pet. Without wishing to be bound by theory, it is believed that, amongst other mechanisms, increasing the concentrations of bifido- and lactic-bacteria in the gastro-intestinal tract of the pet produces nutrients and/or increases the absorption of nutrients which are responsible for the improvement or maintenance of the condition of the skin and coat of the pet. Further, increasing the concentrations of bifido- and lactic-bacteria in the gastro-intestinal tract of the pet are thought to promote a better microflora balance on the skin of the pet. These are believed to contribute to the effective functioning of the skin and coat as an integrated protective unit as well as giving it a flourishing appearance.

The nutritional agent may be a prebiotic or a probiotic micro-organism. Further, both prebiotic fibers and probiotic micro-organisms may be administered to the pet. The prebiotic may also be administered in combination with other active ingredients providing a synergistic benefit to the system or to the coat alone.

Suitable prebiotics include oligosaccharides, such as inulin and its hydrolysis products commonly known as fructooligosaccharides, galacto-oligosaccarides, xylo-oligosaccharides or oligo derivatives of starch.

The prebiotics may be provided in any suitable form. For example, the prebiotic may be provided in the form of plant material which contains the fiber. Suitable plant materials includes asparagus, artichokes, onions, wheat or chicory, or residues of these plant materials. Alternatively, the prebiotic fiber may be provided as an inulin extract. Extracts from chicory are particularly suitable. Suitable inulin extracts may be obtained from Orafti SA of Tirlemont 3300, Belgium under the trade mark “Raftiline”. For example, the inulin may be provided in the form of Raftiline®ST which is a fine white powder which contains about 90 to about 94% by weight of inulin, up to about 4% by weight of glucose and fructose, and about 4 to 9% by weight of sucrose. Alternatively, the fiber may be in the form of a fructooligosaccharide such as obtained from Orafti SA of Tirlemont 3300, Belgium under the trade mark “Raftilose”. For example, the inulin may be provided in the form of Raftilose®P95. Otherwise, the fructooligosaccharides may be obtained by hydrolyzing inulin, by enzymatic methods, or by using micro-organisms.

The probiotic micro-organism may be selected from one or more micro-organisms suitable for animal consumption and which is able to improve the microbial balance in the human or animal intestine.

Examples of suitable probiotic micro-organisms include yeasts such as Saccharomyces, Debaromyces, Candida, Pichia and Torulopsis, moulds such as Aspergillus, Rhizopus, Mucor, and Penicillium and Torulopsis and bacteria such as the genera Bifidobacterium, Bacteroides, Fusobacterium, Melissococcus, Propionibacterium, Enterococcus, Lactococcus, Staphylococcus, Peptostrepococcus, Bacillus, Pediococcus, Micrococcus, Leuconostoc, Weissella, Aerococcus, Oenococcus and Lactobacillus. Specific examples of suitable probiotic micro-organisms are: Saccharomyces cereviseae, Bacillus coagulans, Bacillus licheniformis, Bacillus subtilis, Bifidobacterium bifidum, Bifidobacterium infantis, Bifidobacterium longum, Enterococcus faecium, Enterococcus faecalis, Lactobacillus acidophilus, Lactobacillus alimentarius, Lactobacillus casei subsp. casei, Lactobacillus casei Shirota, Lactobacillus curvatus, Lactobacillus delbruckii subsp. lactis, Lactobacillus farciminus, Lactobacillus gasseri, Lactobacillus helveticus, Lactobacillus johnsonii, Lactobacillus reuteri, Lactobacillus rhamnosus (Lactobacillus GG), Lactobacillus sake, Lactococcus lactis, Micrococcus varians, Pediococcus acidilactici, Pediococcus pentosaceus, Pediococcus acidilactici, Pediococcus halophilus, Streptococcus faecalis, Streptococcus thermophilus, Staphylococcus carnosus, and Staphylococcus xylosus. The probiotic micro-organisms may be in powdered, dried form; especially in spore form for micro-organisms which form spores. Further, if desired, the probiotic micro-organism may be encapsulated to further increase the probability of survival; for example in a sugar matrix, fat matrix or polysaccharide matrix.

The nutritional agent may be administered to the pet as a supplement to the pet's normal diet or as a component of a nutritionally complete pet food. It is preferred to include the nutritional agent in a nutritionally complete pet food. However, if administered as a supplement, this may be done by way of example, by including the agent as in a separate container, such as a sachet in a package together with a nutritionally complete food composition.

The nutritionally complete pet food may be in any suitable form; for example in dried form, semi-moist form or wet form. These pet foods may be produced as is conventional. Apart from the nutritional agent, these pet foods may include any one or more of a starch source, a protein source and lipid source. Suitable starch sources are, for example, grains and legumes such as corn, rice, wheat, barley, oats, soy, and mixtures of these. Suitable protein sources may be selected from any suitable animal or vegetable protein source; for example meat and meat meals, poultry meal, fish meal, soy protein concentrates, milk proteins, gluten, and the like. Suitable lipid sources include meats, animal fats and vegetable fats. The choice of the starch, protein and lipid sources will be largely determined by the nutritional needs of the animal, palatability considerations, and the type of product produced. Further, various other ingredients, for example, sugar, salt, spices, seasonings, vitamins, minerals, flavoring agents, fats and the like may also be incorporated into dried food as desired.

For dried pet foods a suitable process is extrusion cooking, although baking and other suitable processes may be used. When extrusion cooked, the dried pet food is usually provided in the form of a kibble. If a prebiotic is used, the prebiotic may be admixed with the other ingredients of the dried pet food prior to processing. A suitable process is described in European patent application No 0850569. If a probiotic micro-organism is used, the organism is best coated onto or filled into the dried pet food. A suitable process is described in European patent publication Number EP 0 862 863.

For wet foods, the processes described in U.S. Pat. Nos. 4,781,939 and 5,132,137 may be used to produce simulated meat products. Other procedures for producing chunk type products may also be used; for example cooking in a steam oven. Alternatively, loaf type products may be produced by emulsifying a suitable meat material to produce a meat emulsion, adding a suitable gelling agent, and heating the meat emulsion prior to filling into cans or other containers.

The maximum level of prebiotic in the pet food is preferably about 20% by weight; especially about 10% by weight. For example, the prebiotic may comprise about 0.1% to about 5% by weight of the pet food. For pet foods which use chicory as the prebiotic, the chicory may be included to comprise about 0.5% to about 10% by weight of the feed mixture; more preferably about 1% to about 5% by weight.

If a probiotic micro-organism is used, the pet food preferably contains about 10⁴ to about 10¹¹ cells of the probiotic micro-organism per gram of the pet food; more preferably about 10⁶ to about 10⁸ cells of the probiotic micro-organism per gram. The pet food may contain about 0.5% to about 20% by weight of the mixture of the probiotic micro-organism; preferably about 1% to about 6% by weight; for example about 3% to about 6% by weight.

The probiotic may be applied to the pet food in any suitable manner. By way of example, it may be mixed with a suitable carrier substrate and sprayed onto the food particles, such as kibbles. Examples of carrier substrates are edible fats such as tallow or vegetable oils or fats such as hydrogenated soy fat. Protein digests and water may also be used. The carrier may be applied to the pet food prior to the application of the probiotic, or simultaneously.

The pet foods may contain other active agents such as long chain fatty acids and zinc. Suitable long chain fatty acids include alpha-linoleic acid, gamma linolenic acid, linoleic acid, eicosapentanoic acid, and docosahexanoic acid. Fish oils are a suitable source of eicosapentanoic acids and docosahexanoic acid. Borage oil, blackcurrent seed oil and evening primrose oil are suitable sources of gamma linolenic acid. Safflower oils, sunflower oils, corn oils and soy bean oils are suitable sources of linoleic acid. These oils may also be used in the coating substrates referred to above. Zinc may be provided in various suitable forms, for example as zinc sulfate or zinc oxide. Further, many ingredients commonly used in pet foods are sources of fatty acids and zinc. It has been observed that the combination of chicory, as a source of prebiotic, with a linoleic-acid rich oil, such as soy bean oil, provides unexpected benefits, suggestive of a synergistic effect.

The amount of the pet food to be consumed by the pet to obtain a beneficial effect will depend upon the size, type and age of the pet. However an amount of the pet food to provide a daily amount of about 1 g to about 100 g of prebiotic, or about 10⁶ to about 10¹² cells of the probiotic micro-organism, would usually be adequate.

Numerous modifications may be made to the embodiments described above without departing from the scope of the invention. Specific examples are now described for further illustration. The first two involve assessments performed by a trained evaluation panel, the creation of which is now described.

### Evaluation Panel

A trained evaluation panel is created to evaluate the skin and coat condition of pets. Parameters to be evaluated by the panelists are visual parameters, touch parameters, and odor parameters. The panel contains 8 panelists. In order to train the panelists, a full sample range of dogs/cats is selected. Several breeds and colors of dogs or several colors of cats are used. All age groups are included for different levels of coat softness. Similarly, non-odorous and odorous animals are included.

A vocabulary and glossary are then developed together with the panelists. Different animals are considered and a list of all possible descriptors is generated. This is then reduced to the essential terms relating to pet skin and coat parameters. A glossary is then written to define each descriptor. High and low values for each descriptor on the scale are agreed upon.

The panel is then trained. Three cats and three dogs are used. For each attribute, each panelist evaluates the pet on a discrete 7-point scale ranging from “not” to “very”. Each panelist's score is compared to the score of the other panelists. The panelists then agree on how attributes should be scored. The panelists then rate three new dogs/cats for the same attribute. The process is repeated until the scores obtained by the panelists are consistent.

The trained panelists thus:
agree on the descriptors;
have learnt to use an agreed questionnaire and follow test procedures; and
are reasonably consistent.

### Example 1

Four dried pet foods are used in the study. Pet food A is a nutritionally complete dried pet food, available under the brand name ALPO Beefy Dinner. (ALPO is a registered trade mark of SOCIETE DES PRODUITS NESTLE S.A. of SWITZERLAND).

Pet food B is a nutritionally complete dried pet food which is substantially identical to Pet Food A but which contains 3% by weight of linoleic acid. These are the control foods. Pet food 1 is a nutritionally complete dried pet food which is substantially identical to Pet Food A but which contains 2% by weight of chicory. Pet food 2 is a nutritionally complete dried pet food which is substantially identical to Pet Food 1 but which contains 3% by weight of linoleic acid.

A group of 32 dogs are used in the study. The dogs are preferred for 8 weeks using Pet Food A. The dogs are then divided into 4 groups of 8 dogs and fed the following diets for 8 weeks:

| Group | Pet Food Diet |
|---|---|
| A | A |
| B | B |
| 1 | 1 |
| 2 | 2 |

The dogs have free access to water and are fed once a day. The following parameters are determined at 0, 4 weeks, 8 weeks and 12 weeks:
Sebum lipids;
Skin lipids;
Sensory parameters such as skin gloss, scaliness, odor, softness, etc (using the trained panel);
Skin hydration using a corneometer—made in three skin locations (back, below the breast bone, lower abdomen, flank);
Sebum production using a sebumeter—made in three skin locations (back, below the breast bone, lower abdomen);
Skin pH using a skin pH meter—made in three skin locations (back, below the breast bone, lower abdomen);
Transepidermal water loss using a tewameter—made in three skin locations (back, below the breast bone, lower abdomen);
Skin elasticity using a cutometer—made in three skin locations (back, below the breast bone, lower abdomen);
Skin thickness using calipers—made in three skin locations (back, below the breast bone, lower abdomen);
Skin and coat odor;
Skin light reflectance and coat gloss (top of head, base of neck, flank and rump);
oxidative stress such as blood superoxide dismutase levels, glutathione peroxidase and total plasma antioxidant levels;
serum alpha-1-glycoprotein as an inflammation marker;
Blood fatty acids.

The dogs are groomed prior to evaluation by the panel and the panel members do not compare notes during the evaluation.

The skin and coat condition of all dogs is visually and tactilely good as can be expected of normal, healthy dogs. However, the dogs which are fed Pet Foods 1 and 2 have increased coat shininess, coat softness, skin hydration score, skin elasticity; and blood, skin and sebum linoleic content. Further these dogs have reduced transepidermal water loss, oxidative stress and inflammation. All dogs have substantially the same blood chemistry, skin thickness, skin odor, sebum production and skin pH.

Pet Foods 1 and 2 have substantially the same product stability and palatability as Pet Foods A and B.

### Example 2

The procedure of example 1 is repeated with 32 elderly dogs. Four dried pet foods are used in the study. Pet food A is a nutritionally complete dried pet food (ALPO Beefy Dinner). Pet food 3 is a nutritionally complete dried pet food which is substantially identical to Pet Food A but which contains 2% by weight of chicory, 0.5% by weight of alpha-linoleic acid and 3% by weight of linoleic acid. Pet food 4 is a nutritionally complete dried pet food which is substantially identical to Pet Food 3 but which contains 0.5% by weight of eicosapentanoic acid and docosahexanoic acid in place of the alpha-linoleic acid. Pet food 5 is a nutritionally complete dried pet food which is substantially identical to Pet Food 3 but which contains 0.5% by weight of gamma-linolenic acid in place of the alpha-linoleic acid.

The dogs are preferred for 8 weeks using Pet Food A. The dogs are then divided into 4 groups of 8 dogs and fed the following diets for 8 weeks:

| Group | Pet Food Diet |
|---|---|
| A | A |
| 3 | 3 |
| 4 | 4 |
| 5 | 5 |

The evaluation panel's findings are that the skin and coat condition of all dogs is visually and tactilely good as can be expected of normal, healthy dogs. However, the dogs which are fed Pet Foods 3, 4 and 5 have increased coat shininess, coat softness, skin hydration score, skin elasticity; and blood, skin and sebum linoleic content. Further these dogs have reduced transepidermal water loss, oxidative stress and inflammation. All dogs have substantially the same blood chemistry, skin thickness, skin odor, sebum production and skin pH. The dogs fed Pet Food 5 have slightly better skin and coat condition than those fed Pet Foods 3 and 4.

Pet Foods 3, 4 and 5 have substantially the same product stability and palatability as Pet Food A.

### Example 3

Four canned pet foods are used in the study. As in example 1, Pet food “A” is a nutritionally complete dried pet food, available under the brand name MIGHTY DOG Senior Beef and Rice (MIGHTY DOG is a registered trade mark of SOCIETE DES PRODUITS NESTLE S.A. of SWITZERLAND).

Pet food 1 is a nutritionally complete canned pet food which is substantially identical to Pet Food A but which contains 1% by weight of chicory. Pet food 2 is a nutritionally complete canned pet food which is substantially identical to Pet Food A but which contains 0.75% by weight of soybean oil. Pet food 3 is a nutritionally complete canned pet food which is substantially identical to Pet Food A but which contains 1% by weight of chicory and 0.75% by weight soybean oil.

A group of 32 dogs are used in the study. The dogs are preferred for 8 weeks using Pet Food A. The dogs are then divided into 4 groups of 8 dogs and fed the following diets for 12 weeks:

| Group | Pet Food Diet |
|---|---|
| A | A |
| 1 | 1 |
| 2 | 2 |
| 3 | 3 |

The dogs have free access to water and are fed at least once a day. The prevalence of dandruff in the coat is determined by a 30 member evaluation panel at the end of the study.

The dogs are groomed prior to evaluation by the panel and the panel members do not compare notes during the evaluation.

In this evaluation the dogs are presented to each of the individual panelists in 24 different pairings. The panelists are asked to indicate on a their scoresheets which dog of the pair presented displays less dandruff.

The overall coat condition of all dogs is visually and tactilely good as can be expected of normal, healthy dogs. However, the dogs which are fed Pet Food 3 are found to have noticeably less dandruff than those fed on the control diet, suggesting a synergistic effect between the prebiotic chicory and the fatty acids derived from the soybean oil.

### Example 4

A feed mixture is made up of corn, corn gluten, chicken and fish meal, salts, vitamins and minerals. The feed mixture is fed into a preconditioner and moistened. The moistened feed leaving the preconditioner is then fed into an extruder-cooker and gelatinised. The gelatinised matrix leaving the extruder is forced through a die and extruded. The extrudate leaving the die head is cut into pieces suitable for feeding to dogs, dried at about 140° C. for about 20 minutes, and cooled to form pellets. The water activity of the pellets is about 0.6.

The pellets are sprayed with a coating substrate comprising tallow fat. The probiotic, Bacillus coagulans, is in the form of powdered endospores and is applied by dry spraying before the tallow sets so as to adhere to or be partially penetrated in the fat layer. B. coagulans in endosporic form is obtainable from Sankyo Pharmaceutical Company under the trade name Lacris-S. The pellets thus hold about 1.6×10⁶ cells/g of B. coagulans. Results from storage at 37° C. for 8 weeks indicate that the micro-organisms display excellent stability and are likely to be stable after one year of storage at normal conditions.

A trial is conducted using 30 dogs. The dogs are fed a standard dried diet corresponding to diet A in example 3 for a week prior to commencement of the trials. Immediately prior to commencement of the trials, the coat condition of the participating dogs is assessed by an evaluation panel as described in Example 3.

The dogs are then separated into two groups of 15 dogs. One group of dogs is fed the dried pellets coated with B. coagulans and the tallow coating. The other group of dogs continues to be fed diet A, thus providing a control diet. Both groups are given free access to the food and to water.

After 12 weeks, the coat condition of each dog is again evaluated. The dogs which are fed the pellets with the probiotic and tallow coating have a significantly shinier appearance and display no noticeable dandruff than the dogs on diet A.

It should be understood that various changes and modifications to the presently preferred embodiments described herein will be apparent to those skilled in the art. Such changes and modifications can be made without departing from the spirit and scope of the present subject matter and without diminishing its intended advantages. It is therefore intended that such changes and modifications be covered by the appended claims.

The invention is claimed as follows:

1. A method for treating a pet, the method comprising feeding the pet a food composition comprising a nutritional agent comprising about 1% to about 2% chicory by weight of the food composition, a probiotic micro-organism, and about 0.75% to about 3% linoleic acid by weight of the food composition.

2. The method of claim 1, wherein the nutritional agent further comprises, by weight of the food composition, about 0.5% of a long chain fatty acid selected from the group consisting of about alpha-linoleic acid, a combination of eicosapentanoic acid and decosahexanoic acid, and gamma linolenic acid.

3. The method of claim 1, wherein the nutritional agent comprises about 0.75% linoleic acid by weight of the food composition.

4. The method of claim 1, wherein the linoleic acid has a source selected from the group consisting of safflower oil, sunflower oil, corn oil and soybean oil.

5. The method of claim 4, wherein the source is soybean oil.

6. The method of claim 1, wherein the nutritional agent is administered as a supplement to the pet's normal diet.

7. The method of claim 1, wherein the nutritional agent further comprises a zinc source.

8. The method of claim 1, wherein the pet food contains about 10⁴ to about 10¹¹ cells of a probiotic micro-organism per gram of the pet food.

9. A method for improving or maintaining shininess and softness of the coat of a pet, the method comprising administering to the pet in need of same a food composition comprising a nutritional agent that increases the digestion of nutrients in the gastro-intestinal tract of the pet, wherein the nutritional agent comprises about 1% to about 2% chicory by weight of the food composition, a probiotic micro-organism, and about 0.75% to about 3% linoleic acid by weight of the food composition.

10. A method of reducing or assisting in the prophylaxis of dandruff in the coat of a pet, the method comprising administering to the pet in need of same a food composition comprising a nutritional agent that improves the microflora balance on the skin of the pet, wherein the nutritional agent comprises about 1% to about 2% chicory by weight of the food composition, a probiotic micro-organism, and about 0.75% to about 3% linoleic acid by weight of the food composition.

11. A method of manufacturing a pet food, the method comprising providing about 1% to about 2% chicory by weight of the pet food, a probiotic micro-organism, and about 0.75% to about 3% linoleic acid by weight of the pet food for giving the coat of a pet animal eating it a flourishing appearance.

12. A method of reducing dandruff in a coat of a pet animal, the method comprising providing a pet food composition comprising about 1% to about 2% chicory by weight of the pet food composition, a probiotic micro-organism, and about 0.75% to about 3% linoleic acid by weight of the pet food composition and administering the pet food composition to the pet animal in need of same.
